(19) United States
(12) Patent Application Publication
(10) Pub. No.: US 2007/0199478 A1
(43) Pub. Date: Aug. 30, 2007

(54) Thin Covering Aluminum Pigments, Process For The Production Thereof, And Use Of Said Aluminum Pigments

(75) Inventors: Thomas Schlegl, Simmelsdorf (DE); Stefan Trummer, Nurnberg (DE); Frank Henglein, Nurnberg (DE); Ralph Schneider, Lauf (DE); Thomas Schuster, Lauf (DE)

Correspondence Address: Richard C Woodbridge;Synnestvedt Lechner & Woodbridge, 112 Nassau Street, PO Box 592, Princeton, NJ, 08542-0592, US

(73) Assignee: Eckart GmbH & Co., KG, Furth (DE)

(21) Appl. No.: 10/552,116

(22) PCT Filed: Apr. 2, 2004
(86) PCT No.: PCT/EP04/03553

§ 371 Date: Nov. 2, 2006

(30) Foreign Application Priority Data
Apr. 4, 2003 (DE) 103 15 775.1

(51) Int. Cl. C09D 11/00; A61K 8/00; C09C 1/62

(52) U.S. Cl. 106/404; 106/31.9; 424/61; 424/401

(57) ABSTRACT

The invention relates to aluminum pigments which are at least partially coated with lubricants, wherein said aluminum pigment have
a) a water coverage between 40,000 and 130,000 cm²/g,
b) a mean thickness h of less than 100 to 30 nm, as calculated from the water coverage as well as the h₅₀ value determined from the cumulative breakthrough curve of a scanning electron microscope thickness count,
c) as determined by a scanning electron microscope thickness count, a relative width of the distribution of thicknesses Δh between 70% and 140%, which is calculated on the basis of the corresponding cumulative breakthrough curve of the relative frequencies according to the formula

$$Δ h = 100 \times \frac{{h}_{90} - {h}_{10}}{{h}_{50}} , ,$$
d) an aspect ratio d₅₀/h of more than 200, e) a roughness value of from 0.30 to 0.9, calculated from the specific surface area, as determined by the BET test method, and the water coverage according to the following formula BET value/2×water coverage. The invention also relates to a process for the manufacture of said aluminum pigments and to the use thereof.

The invention relates to aluminum pigments which are at least partially coated with lubricants, and to a process for the production thereof. The invention furthermore relates to uses of the aluminum pigments.

Aluminum pigments are effect pigments and are distinguished by their unique metallic appearance and their high covering power. On account of the lamellar structure of these effect pigments they orient themselves in the coating medium parallel to the substrate and cause a metallic effect due to a combination of many individual tiny mirrors. This metallic effect is very strongly pronounced, in particular in wet lacquers. In the case of full-tone lacquers, the result is due to the brightness effect dependent on the angle of observation and/or angle of incidence, which is also referred to as “flop”. Good flop is influenced by many properties of the pigments: thus their orientation, their size and size distribution, their surface texture (roughness) and the edge texture all play an important part.

The driving force for a plane-parallel orientation of the pigments, which are also referred to as flakes—in addition to surface chemistry incompatibilities of the aluminum pigments and the binder system—is especially the aspect ratio. The aspect ratio is understood as meaning the ratio of the longitudinal extent d to the thickness h of the pigments. Longitudinal extent is mainly determined by laser scattering methods. In this case, the d₅₀ value of the cumulative breakthrough curve is usually used.

Since the longitudinal extent of the aluminum pigments is strongly dependent on the respective intended use, a high aspect ratio and thus the best possible orientation can especially be achieved by way of the thickness of the pigments. Thin pigments orient themselves better and therefore have higher flop.

A further important characteristic of metallic coatings or printing inks is their high gloss. Gloss, inter alia, is a physiologically and psychologically related variable, but according to DIN 67 530, the “gloss power” of a plane surface is recorded by reflectometer values. The reflection in the gloss angle is measured relatively to a standard (as a rule a black mirror glass plate). According to this standard, highly glossy samples (reflectometer value >70) are measured at an angle of incidence or reflection of 20° and medium glossy surfaces at 60°. A prerequisite for a good gloss of metallic coatings is likewise a maximum plane-parallel orientation of the lamellar pigments in the coating medium.

The most brilliant aluminum pigments with the highest gloss and flop are at present assigned to two classes: on the one hand, “silver dollar pigments”, which are prepared by wet grinding of aluminum shot, and, on the other hand, “PVD pigments”. Silver dollar pigments are distinguished from metal pigments obtained by comminutive grinding by a relatively round shape and a relatively smooth surface.

Aluminum pigments prepared by wet grinding and with a high degree of reflection and high coverage are described, for example, in EP 0 451 785 B2. The pigments are characterized by water coverages (spreading values) of from 2.5 to 5.0 m²/g, a roughness value of 2.0 or less and an aspect ratio d₅₀/h of 90 or more. Aspect ratios of up to a maximum of 140 are disclosed in the examples of EP 0 451 785 B2.

EP 0 451 785 B2 also notes that the covering power of aluminum pigments is dependent not only on their longitudinal extent, but in particular also on their thickness. Thinner pigments in this case exhibit a higher covering power.

Aluminum pigments for automobile lacquers typically exhibit d₅₀ values of from 15 to 20 μm. An aluminum pigment according to the teaching of EP 0 451 785 B2 prepared with a water coverage of 5.0 m²/g and an aspect ratio of 90 would have a mean thickness h of 80 nm and therefore a d₅₀ value of 7.2 μm. A pigment of this type would be too small, for example, for automobile lacquers.

Aluminum pigments having d₅₀ values of from 15 to 20 μm customary for this market segment and an aspect ratio of 90 would have a mean thickness h in the range of from 167 to 222 nm.

In U.S. Pat. No. 4,318,747, fine aluminum pigments having an average size of less than 5 μm with leafing character are disclosed, which possess a water coverage of at least 50,000 cm²/g, and a specific surface area, measured according to the BET method, of from 24 m²/g to 93 m²/g. From these data, roughness values in the range of from 2.4 to 9.3 can be calculated.

On account of the great roughness of the surface of these pigments, a strong scattering of irradiated light and consequently a reduced gloss occurs, compared with the smooth surface of a PVD pigment.

Furthermore, neither the aluminum pigments disclosed in EP 0 451 785 B2 nor the aluminum pigments disclosed in U.S. Pat. No. 4,318,747 are suitable for use in, say, automobile lacquers on account of their fineness.

The examples described in U.S. Pat. No. 4,318,747 are always ground for long grinding times with steel balls of 5 mm diameter. The use of balls of this type is typical of comminution processes.

In U.S. Pat. No. 3,776,473, aluminum pigments having a high reflectivity and a smooth surface and a round shape are described. The pigments referred to in this patent in the exemplary embodiments exhibit water coverages of not more than only 15,600 cm²/g.

With PVD pigments, extremely thin (thicknesses: 20 to 50 nm) Al pigments are prepared. The distribution of thicknesses of these pigments is extremely low. In this process aluminum is evaporated in an ultrahigh vacuum on to a carrier foil provided with a release-coat. This release coat is as a rule a polymer. Subsequently the evaporated aluminum is separated—as far as possible—from the carrier foil in a solvent and the metal foil is comminuted mechanically or by means of ultrasound. The production of PVD pigments is described, for example, in J. Seubert and A. Fetz, “PVD Aluminum Pigments: Superior Brilliance for Coatings and Graphic Arts”, Coatings Journal, Vol. 84, A6 225-264, July 2001, pages 240-245.

On account of their extreme thinness, these PVD pigments exhibit excellent covering power. The thin pigments are so flexible that they virtually “cling” to the undercoat. To display their optical possibilities they should therefore be applied to a smooth undercoat.

The extremely high production costs of the preparation process, however, are a drawback of these PVD pigments. It is furthermore a disadvantage that the release coat can barely be completely removed from the pigment particles. This adherent polymer film can, however, lead to disadvantages. Thus, in the case of a printing ink, incompatibilities with the solvent used in the printing ink can occur. For example, polymer films which are suitable for toluene can be incompatible in solvents such as alcohols or water. This becomes noticeable in the formation of agglomerates, which completely destroy the desired decorative effect.

In particular, however, polymeric adhesions of this type can have a deleterious effect if the aluminum pigments are provided, after production thereof, with chemical protective coatings, such as are described, for example, in DE 196 35 085, in order to make them corrosion-resistant.

The same applies to stabilization by anticorrosive agents, such as are described, for example, in DE 100 01 437. Here, residues of adherent release-coat lead to an uneven protective coating and prevent the coating of a reproducibly prepared protective layer. In particular, the use of coated substrates of this type in water lacquers, in which unstabilized aluminum pigments produce undesirable gassing caused by evolution of hydrogen, is not reproducibly achievable with precoated substrates of this type.

A further serious drawback is that the PVD pigments exhibit an extremely strong tendency to agglomerate. For this reason, PVD pigments are only supplied in highly dilute dispersions with an aluminum pigment content of usually 10% by weight. To achieve ease of handling it is desirable to have preparations having a higher aluminum pigment content.

It is an object of the present invention to provide very thin aluminum pigments without adherent polymer film and with excellent covering power, high gloss and, compared with conventional aluminum pigments obtained by conventional wet grinding, improved metallic appearance, the “chrome effect”.

It is a further object of the invention to provide very thin aluminum pigments having a markedly reduced tendency to agglomerate compared with PVD pigments.

Furthermore, pigments of this type should be capable of being prepared by a more cost-effective process, compared with the expensive PVD production process.

This object is achieved by the provision of aluminum pigments, which are at least partially coated with lubricants, and which have
a) a water coverage between 40,000 and 130,000 cm²/g,
b) a mean thickness h of less than 100 to 30 nm as calculated from the water coverage and the h₅₀ value as determined from the cumulative breakthrough curve of a scanning electron microscope thickness count,
c) as determined by a scanning electron microscope thickness count, a relative width of the distribution of thicknesses Δh of from 70% to 140%, as calculated on the basis of the corresponding cumulative breakthrough curve of the relative frequencies of occurrence, according to the formula

$$Δ h = 100 \times \frac{{h}_{90} - {h}_{10}}{{h}_{50}} ,$$
d) an aspect ratio d₅₀/h of more than 200,
e) a roughness value of from 0.30 to 0.9, as calculated from the specific surface area as determined by the BET test method and the water coverage, according to the formula
   BET value/2×water coverage.

Preferred developments of the aluminum particles of the invention are defined in the subclaims.

The object underlying the invention is furthermore achieved by a process for the production of aluminum pigments according to any one of claims 1 to 15, which comprises the following step:
a) grinding of aluminum particles using a grinder in the presence of solvent and lubricants and grinding media having an individual weight of from 2 to 13 mg, over a period of from 15 to 72 hours to produce aluminum pigments.

Preferred developments of the process are defined in the subclaims.

The object of the invention is further achieved by the use defined claims 23 or 24, and a nail varnish defined in claim 25 as also a water lacquer define in claim 26.

This invention relates to aluminum pigments, which are at least partially coated with lubricants, have a water coverage of from 40,000 to 130,000 cm²/g, a mean thickness h of less than 30 nm as calculated from the water coverage and the h₅₀ value as determined from the cumulative breakthrough curve of a scanning electron microscope thickness count, a width of the distribution of thicknesses of from 70% to 140%, as determined by a scanning electron microscope thickness count, which is calculated with the aid of the cumulative breakthrough curve according to the formula

$$Δ h = 100 \times \frac{{h}_{90} - {h}_{10}}{{h}_{50}} ,$$

and an aspect ratio d₅₀/h over 200.

The preferred aluminum pigments of the invention have a water coverage of from 45,000 to 125,000 cm²/g and a mean thickness h of less than 89 to 32 nm calculated from the water coverage, and as determined by a scanning electron microscope thickness count (h₅₀ value of the cumulative breakthrough curve). Other preferred aluminum pigments of the invention have a water coverage of from 50,000 to 120,000 cm²/g, preferably 50,000 to 90,000 cm²/g, and a mean thickness h of less than 80 to 33 nm, preferably less than 80 to 44 nm, as calculated from the water coverage, and as determined by a scanning electron microscope thickness count (h₅₀ value of the cumulative breakthrough curve).

The aluminum pigments of the invention possess a very high covering power on account of their low thickness.

Poor stacking of the pigments in a coating medium is particularly detrimental to obtaining good orientation of aluminum pigments. In order to achieve stacking of the pigments which is as even as possible in the coating medium, the thin pigments should advantageously have a narrow distribution of thicknesses and a low pigmentation level.

With the conventional thick aluminum pigments and with a wide distribution of thicknesses uneven stacking of the pigments readily occurs. Thus, very thick pigments can, in particular, serve as “spacers” and therefore adversely affect the orientation (gloss) and the covering power of the surrounding pigments.

The aluminum pigments of the invention are surprisingly very thin and at the same time have a narrow distribution of thicknesses. The aluminum pigments of the invention are surprisingly similar in their optical properties to the PVD pigments, but,—compared with the expensive PVD process—are significantly easier to prepare and exhibit strongly improved handling properties, which, for example, allow for a significantly higher concentration in preparations.

The exact mean thickness of lamellar metal pigments can be determined only with great difficulty. DIN 55923 specifies a procedure for the measurement of the water coverage (spreading) of “leafing” pigments.

Here, a defined weight of aluminum pigments in a readily volatile organic solvent is placed on a water surface in a trough. As leafing pigment, the aluminum pigment is coated, for example with stearic acid, and thereby strongly hydrophobized. The pigments spread out on the water surface and form a silver metal film. By stirring with a glass rod, they are dispersed to give a uniform “cloudless” metal film. Subsequently, the film is pushed together by two rulers, until it exhibits its first wrinkles. The film is then expanded again until the wrinkles disappear. The area covered by the metal film is measured and defined as the water coverage in cm²/g (or alternatively in m²/g) based on the weight of the pigment.

In this method, it is assumed that the metal pigments are arranged next to one another, at least on average, and are consequently present in a single “monolayer” of pigment.

With the aid of this water coverage, the mean thickness h in nm of the pigments is calculated according to the following formula:

$$h = \frac{{10}^{7} ( nm / cm )}{ρ ( g / {cm}^{3} ) * water coverage ( {cm}^{2} / g )}$$

where ρ is the physical absolute density of the pigment adsorbed with stearic acid. A value of about 2.5 g/cm³ is usually measured here.

In the above DIN standard, only the investigation of leafing pigments is provided for. Conventional nonleafing pigments can also be measured according to this method if they have been coated with stearic acid before spreading. With the aid of the water coverage, only a mean thickness h of the pigments can be determined, but this method is unable to give information on the width of the distribution of thicknesses.

The thickness of the pigments can likewise be determined with the aid of a scanning electron microscope (SEM). Here, a sufficient number of particles should be measured so as to provide a representative mean value. Customarily, approximately 100 particles are measured. With this process—in contrast to the water spreading method—an overview of the distribution of thicknesses of the pigments is also obtained.

The h₅₀ value of the thickness cumulative breakthrough curve presents itself as a mean value. A measure of the width of the distribution Δh is indicated by the following formula:

$$Δ h ( \% ) = 100 * \frac{{h}_{90} - {h}_{10}}{{h}_{50}}$$

where the indices relate to the respective value of the cumulative breakthrough curve.

The pigments of the invention possess a relative width of the distribution of thicknesses Δh of from 70 to 140%.

Preferably, the pigments of the invention possess a relative width of the distribution of thicknesses Δh of from 75 to 120%.

The aspect ratio f is understood as meaning the ratio of the mean value of the longitudinal extent to the mean thickness of the aluminum pigment platelets.

The longitudinal extent d (diameter) is determined in laser scattering experiments on the basis of the Fraunhofer and/or the Mie diffraction theory. The evaluation of the diffraction data is based on a model which aims at the diameter of an equivalent sphere. No absolute values are therefore obtained, however the diameters measured have gained acceptance as reliable relative values for the description of the size characteristics of lamellar metal pigments.

The dimensionless aspect ratio f is then defined as:

$$f = 1000 * \frac{{d}_{50} ( μ m )}{h ( nm )}$$

The d₅₀ value here corresponds to 50% of the cumulative breakthrough curve, measured and evaluated in the form of a volume distribution of equivalent spheres.

A further variable for the pigment characterization is the dimensionless roughness value R. This is understood as meaning the ratio of the specific surface area, measured according to the BET method (DIN 66132), to the geometric pigment surface area. The latter can be calculated, neglecting the edge of the pigments, as twice the water coverage:

$$R = \frac{BET value ( {m}^{2} / g ) * {10}^{4}}{2 * water coverage ( {cm}^{2} / g )}$$

The roughness value is likewise to be considered as a relative value, since the two surface determination methods do not yield exact results. An ideal smooth surface should theoretically possess a roughness value of 1. In fact, however, values of less than 1 are occasionally found.

In the longitudinal extent, the aluminum pigments of the invention do not differ fundamentally from aluminum pigments conventionally found on the market which have been prepared by wet grinding. Specifically, the sizes depend in individual cases on the intended use. The d₅₀ values of the distribution of lengths are preferably above 6 μm, more preferably in a range of from 6 μm to 50 μm, even more preferably from 8 μm to 45 μm, even more preferably from 12 μm to 40 μm, very preferably from 15 μm to 30 μm, and most preferably from 20 μm to 25 μm.

The pigments of the invention are distinguished by an aspect ratio f of more than 200. Preferably, the pigments of the invention possess an aspect ratio f of more than 220, more preferably more than 240, and most preferably more than 300. According to a further preferred embodiment, the aluminum pigments exhibit a roughness value R of from 0.35 to 0.9, more preferably of from 0.4 to 0.8.

These values show that they are very thin pigments with relatively smooth surfaces.

A comparatively low content of active aluminum is furthermore characteristic of the pigments of the invention.

This content can be determined by completely dissolving a defined amount of aluminum pigments in an alkaline solution and recording the resulting hydrogen volumetrically under temperature-controlled conditions. In the case of these pigments, said content lies in a range of from 85 to 93, preferably from 87 to 92%, based on the total weight of the aluminum pigments. This is to be compared with values of 93 to 97% for conventional pigments in the case of aluminum pigments obtained by wet grinding.

The residual content in the pigment can be attributed to aluminum oxide, and to fatty acids bound to the surface. On account of the low thickness of the aluminum pigments of the invention, they possess a comparatively high relative oxide content. The content of fatty acids is also comparatively high. The latter can be roughly estimated from the C content determined by elemental analysis. In the case of the pigments of the invention, the residual content is typically from 0.7 to 1.5% by weight, preferably from 0.8 to 1.4% by weight, as measured on aluminum powders previously washed with acetone or comparable solvents and subsequently dried.

The aluminum pigments of the invention are very thin pigments with a narrow distribution of thicknesses. Pigments of this type possess a high covering power. The aluminum pigments of the invention preferably exhibit a distribution of thicknesses having a d₉₅ value of less than 200 nm, preferably less than 150 nm. The narrow distribution of thicknesses advantageously causes very good stacking of the pigments in a coating medium, for example, a lacquer or a printing ink. With the pigments of the invention, for example, it is possible to obtain good covering effects with very high gloss and very good flop by applying very low layer thicknesses, for example a layer thickness of less than 10 μm.

Particularly in automobile lacquering, there is a need for lower layer thicknesses. The driving force here is especially a saving in cost. Hitherto, base lacquer layer thicknesses have been typically in the region of 15 Ξm. Even now, on very curved shaped parts, such as door handles, lower layer thicknesses are routinely used. It would be desirable if low layer thicknesses down to less than 10 μm could be realized. However, the layer thickness may not be too low, as otherwise problems of adhesion, coverage and/or pigmentation will arise.

The pigments of the invention are extremely suitable for use in coating compositions which are intended to be applied in very thin layer thicknesses.

In the following, the process for the production of the aluminum pigments of the invention will be described. This is distinguished by an extremely gentle formative grinding of aluminum particles. Specifically, the process consists of the following steps:

The aluminum particles are ground using a grinder, preferably a ball mill, stirred ball mill, or an attritor, in the presence of solvents and lubricants as grinding aids, and of grinding media which individually weigh from 2 to 13 mg, over a period of from approximately 15 hours to approximately 72 hours.

According to a preferred development of the invention, the grinding media individually weigh from 5.0 to 12.0 mg. As grinding media, preferably spherical media, and more preferably balls, are used.

After the aluminum particles have been ground, the aluminum pigments obtained are separated from the grinding media, preferably the grinding spheres. In a further process step, the aluminum pigments obtained can be subjected to size classification. Subsequently, the aluminum pigments can be converted into a suitable supply form. For example, the classified or the unclassified aluminum pigments can be converted to a powder form, preferably a nondusting powder form. The aluminum pigments may alternatively be converted to a paste, granules, or pellets by compaction.

For the purposes of the invention, pellets are regarded as including briquettes, tablets, or small cylinders.

Pelletization can be carried out on a pelletizing plate in conventional manner. Tableting can take place in a tableting device. The small cylinders can be prepared by a molding process for aluminum paste or powder or by extruding an aluminum paste through an extruder and by chopping the extruded strings of paste by means of a rotating knife arrangement. Granulation of the aluminum pigments of the invention can be carried out, for example, by spray granulation.

The aluminum pigments of the invention can be extremely advantageously provided in the form of granules or pellets having high aluminum pigment contents of, say, from 98% by weight to 50% by weight and preferably from 95% by weight to 70% by weight. The aforementioned preparations can be very readily incorporated, for example, into lacquer systems or printing inks without the occurrence of undesirable agglomeration of the aluminum pigments.

Grinding can take place in a solvent in a weight ratio of solvent to aluminum particles of from 2.8 to 10 and in a weight ratio of the grinding spheres to aluminum particles of from 20 to 70 and using lubricants as grinding aids.

The critical speed of rotation n_{crit} is an important parameter which indicates when the balls begin to press against the mill wall due to centrifugal forces, at which point virtually no more grinding takes place:

$${n}_{crit} = \sqrt{\frac{g}{2 {π}^{2}} \cdot \frac{1}{D}}$$

where
D is the diameter of the drum and
g is the gravitational constant.

The speeds of rotation of the ball mill are preferably from 25% to 68% and more preferably from 50% to 62% of the critical number of revolutions n_{crit}.

Low speeds of rotation favor slow transformation of the aluminum particles. In order to cause slow transformation, light grinding spheres are preferably used in the process of the invention. Grinding spheres individually weighing more than 13 mg transform the aluminum particles too vigorously, which leads to premature breakage thereof. The aluminum particles used preferably consist of aluminum shot.

The conditions referred to above lead to very gentle grinding, in which the aluminum particles are slowly shaped and breakages which could result from ball impacts involving high kinetic energy are avoided. On account of the extremely gentle manner of grinding, this type of grinding takes a comparatively long time. The grinding time is from 15 to 72 h, preferably from 16 to 50 h.

The long grinding times lead to a large number of pigment-ball impacts. As a result, the pigment is very uniformly shaped, which is manifested by a very smooth surface and a narrow distribution of thicknesses.

In contrast to conventional grinding processes, the aluminum particles in the process of the invention are for the major part not ground or comminuted, but transformed extremely gently over a relatively long period.

The aluminum grinding material used is preferably aluminum shot. This aluminum shot is preferably prepared in atomizers by atomization of liquid aluminum. Foil powder from an aluminum foil and waste foils can be used. The shot can have a round or irregular shape. Aluminum particles in needle form are not used as starting material in the process of the invention, since these cannot be ground to give thin effect pigments. It is preferred that the aluminum particles have a spherical to ellipsoidal shape.

The aluminum shot should preferably have a mean diameter of less than 100 μm and preferably less than 30 μm, more preferably less than 20 μm, and most preferably less than 10 μm. The purity of the aluminum used is preferably from 99.0 to better than 99.5%.

As lubricants, a large number of compounds can be used. In this connection, mention may be made of the fatty acids containing alkyl radicals having from 10 to 24 carbons already in use for many years. Preferably, stearic acid, oleic acid, or mixtures thereof are used. When stearic acid is used as a lubricant, leafing pigments are formed. Oleic acid, on the other hand, leads to nonleafing pigments. Leafing pigments are characterized in that they are buoyant in a coating medium, such as a lacquer or a printing ink, i.e. they float on the surface of the coating medium. Nonleafing pigments arrange themselves, on the other hand, within the coating medium. Long-chain amino compounds, for example, can additionally be added to the fatty acids. The fatty acids can be of animal or vegetable origin. Likewise, organic phosphonic acids and/or phosphoric acid esters can be used as lubricants.

The lubricant should not be employed in too small an amount, since otherwise the vigorous transformation of the aluminum particles can lead to very large surface areas of the prepared lamellar aluminum pigments that are only inadequately saturated by adsorbed lubricant. In this case, cold welding occurs. Typical amounts are therefore 1 to 20% by weight, preferably 2 to 15% by weight, of lubricant based on the weight of aluminum employed.

The choice of solvent is not critical as such. It is possible to employ customary solvents such as white spirit, solvent naphtha, etc. The use of alcohols, such as, for example, isopropanol, ethers, ketones, esters, etc. is possible.

Likewise, water (at least as the major proportion) can be used as a solvent. In this case, the lubricants employed, however, should have markedly anticorrosive action. Phosphonic acids and/or the phosphoric acid esters, which can also carry ethoxylated side chains, are preferred. The addition of corrosion inhibitors during the grinding process is also advantageous.

The balls preferably used preferably weigh individually from 2 to 13 mg. More preferably, the balls preferably used have an individual weight of from 5.0 to 12.0 mg. Balls having a smooth surface, as round a shape as possible and a uniform size are preferred. The ball material can be steel, glass or ceramics, such as, for example, zirconium oxide or corundum.

The temperatures during the grinding process are in the range of from 10° C. to 70° C. Temperatures ranging from 25° C. to 45° C. are preferred.

Due to the preparation process of the invention, the aluminum pigments of the invention are free from adherent polymer foils, which is a great advantage. The aluminum pigments of the invention therefore do not suffer from the disadvantages of aluminum pigments still encumbered with residues of the release coats, such as are prepared in PVD processes. Moreover, their manner of production is cheaper than the expensive PVD production processes. The separation of the resulting aluminum pigments from the grinding media, preferably grinding spheres, can be carried out in conventional manner by screening.

Following separation of the grinding spheres, the aluminum pigments are preferably subjected to a size classification. This classification should be carried out gently, in order not to destroy the thin aluminum pigments. It may involve, for example, wet screening, decantation, or alternatively separation by sedimentation. In wet screening, the coarse fraction is usually screened off. In the other processes, the fines, in particular, can be separated. Subsequently, the suspension is freed from excess solvent (for example with the aid of a filter press).

In the last step, further processing takes place to give the desired supply form. This can comprise replenishment with a solvent, to give a paste, or drying, to yield a powder.

The dried powder can be further processed by the addition of very small amounts of solvent (<10%) in a suitable homogenizer to give a nondusting metal powder. The filter cake may be first dried out and subsequently made into a paste again with another solvent (preferential wetting).

Finally, the pigments of the invention can be further processed by treating the filter cake with a suitable dispersion of a suitable resin to give pellets, granules, or tablets. These supply forms possess the advantages that they do not dust, exhibit easy meterability and are excellently dispersible.

On account of the very high specific surface areas of the aluminum pigments of the invention, relatively large amounts of dispersing resin must be used, for example, for the process of pelletizing the aluminum pigments of the invention.

It is preferred to use from 2 to 50% by weight, more preferably from 5 to 30% by weight, of resin, based on the total formulation of the pellet.

Pelletization may be carried out using a large number of dispersing resins. Examples thereof are both naturally occurring and synthetic resins. They include, for example, alkyd resins, carboxymethyl and carboxyethyl cellulose resins, cellulose acetate, cellulose acetate propionate (CAP) and cellulose acetate butyrate (CAB), coumarol-indene resins, epoxide esters, epoxide-melamine and epoxide-phenol condensates, ethyl and methyl cellulose, ethylhydroxyethyl cellulose, hydroxyethyl cellulose, hydroxypropylmethyl cellulose, ketones and maleic acid resins, melamine resins, nitrocellulose resins, phenol and modified phenol resins, polyacrylamide resins, polycarbonate resins, polyamide resins, polyester resins, polyether resins, polyurethane resins, and vinyl resins.

Of these polymeric resins, mention may be made in particular of acrylate copolymers and acrylic ester resins, polyacrylonitrile resins and acrylonitrile copolymer resins, copolymers of butadiene and vinylidene chloride, butadiene/styrene copolymers, methyl acrylate and methyl methacrylate copolymers; and polybutene resins, polyisobutylene resins, polyvinyl acetate resins, polyvinyl alcohol resins, polyvinyl chloride resins, polyvinyl ether resins, polyvinylpyrrolidone resins, and polystyrene resins. Further copolymers comprise styrene/maleic anhydride resins and styrene/shellac resins, vinyl chloride/vinyl acetate resins, vinyl chloride/vinyl ether resins and vinyl chloride/vinylidene chloride resins.

Naturally occurring resins such as gum arabic, gutta percha, casein, and gelatin are also suitable.

Aldehyde resins such as the Laropal series produced by BASF AG, Ludwigshafen are preferred.

Furthermore, waxes form suitable binder materials. Here, natural waxes such as beeswax, candelilla waxes, carnauba waxes, montan waxes, and paraffin waxes may be mentioned by way of example. Synthetic waxes such as, for example, PE waxes are likewise suitable.

It has been found, surprisingly, that the tendency of the aluminum pigments of the invention to agglomerate is markedly lower than that of PVD pigments.

It is presumed that this effect is due, not only to the thickness of the pigments, but also to the distribution of thicknesses and the roughness of the aluminum pigments of the invention. With a distribution of thicknesses in the range of from 70% to 140%, a strongly reduced tendency to agglomerate occurs. In addition, the aluminum pigments of the invention exhibit a certain measure of roughness or waviness on account of the production process, which prevents plane-parallel adherence, i.e. an agglomeration of aluminum pigments to one another without, surprisingly, there being any significant impairment of the optical properties, such as reflective capacity and gloss, of the aluminum pigments of the invention.

Unlike PVD pigments, superposed aluminum pigments of the invention exhibit, on account of their roughness or waviness, only point-like contact surfaces with one another. As a result—unlike PVD pigments—the formation of short-range forces of attraction, such as van der Waals forces or hydrogen bridges, is minimized and consequently agglomeration or aggregation is hindered.

In a further embodiment of the invention the aluminum pigments of the invention are subsequently covered or coated with a passivating inhibitor and/or anticorrosive layer. Only with coatings of this type is it possible to safely use the pigments of the invention in water lacquers and/or in external coatings.

The mechanism of action of the passivating layers is complex. In the case of inhibitors, it is usually based on steric effects. The major portion of the inhibitors therefore has an orienting action in the direction of leafing or nonleafing, i.e. of being buoyant or nor buoyant in the medium.

The inhibitors are added, inter alia, in low concentrations in the order of magnitude of 0.5% by weight to 15% by weight based on the weight of the aluminum pigments employed.

For inhibition, the following are preferably suitable:

Organically modified phosphonic acids of the general formula R—P (O)(OR₁)(OR₂), where: R=alkyl, aryl, alkylaryl, arylalkyl, and of alkyl ethers, in particular ethoxylated alkyl ethers, and R₁, R₂═H, CₙH₂ₙ₊₁, where n=1 to 6, in which alkyl can be branched or unbranched. R₁ and R₂ can be the same or different.

Organically modified phosphoric acids and esters of the general formula R—O—P(OR₁)(OR₂) in which R=alkyl, aryl, alkylaryl, arylalkyl, and of alkyl ethers, in particular ethoxylated alkyl ethers and R₁, R₂, ═H, CₙH₂ₙ₊₁, in which n=1 to 6 and alkyl can be branched or unbranched.

Pure phosphonic acids or esters thereof or phosphoric acids or esters thereof or any desired mixtures thereof can be used.

In the case of grinding of the aluminum particles in mainly aqueous solvents, inhibitors of this type are used as grinding aids in order to prevent the formation of hydrogen during the grinding process, which would constitute a safety hazard.

Furthermore, the passivating inhibitor layer can consist of or include corrosion-inhibiting organically functionalized silanes, aliphatic or cyclic amines, aliphatic or aromatic nitro compounds, oxygen-, sulfur- and/or nitrogen-containing heterocyclics such as, for example, thiourea derivatives, sulfur and/or nitrogen compounds of higher ketones, aldehydes, and alcohols (fatty alcohols), thiols, β-ketoesters, β-diketones, or mixtures thereof. The passivating inhibitor layer can, however, consist of the aforementioned substances. Organic phosphonic acids and/or phosphoric acid esters or their mixtures are preferred.

Passivation by means of anticorrosion barriers with chemical and physical protective action can be realized in a variety of ways.

Passivating anticorrosion layers which guarantee the aluminum pigments particularly good corrosion protection include or consist of silicon oxide, chromium oxide, which is preferably applied by a chromating process, zirconium oxide, aluminum oxide, polymerized synthetic resins, phosphate, phosphate, or borate, or mixtures thereof.

Silicon oxide layers and chromium oxide layers (chromation) are preferred. The SiO₂ layers are preferably prepared by sol-gel processes with layer thicknesses of from 20 to 150 nm in organic solvents.

The aluminum pigments of the invention are used in coatings, lacquers, printing inks, powder lacquers, plastics and cosmetic formulations. Preferably, the aluminum pigments of the invention are used in nail varnish formulations. The nail varnish of the invention possesses an extremely metallic appearance.

The aluminum pigments of the invention passivated by subsequent coatings are preferably used in water lacquers and external coatings. The water lacquer of the invention contains, in addition to the passivated aluminum pigments of the invention, the customary water-compatible binders such as polyesters, polyacrylates, polymethacrylates and/or polyurethanes, etc.

The following examples serve to explain the invention, without restricting it in any way.

### EXAMPLES 1 TO 3 OF THE INVENTION

### EXAMPLE 1

3.1 kg of glass spheres (diameter: 2 mm), 310 g of white spirit, 93 g of aluminum shot (mean diameter <8 μm), and 9.3 g of oleic acid are placed in a barrel mill (length: 32 cm, width: 19 cm). Subsequently, the mixture is ground at 57 rpm for a period of 20 h. The product is separated from the grinding balls by rinsing with white spirit and subsequently screened in a wet screening process on a 25 μm screen. The fines are largely freed from white spirit by means of a suction filter and subsequently made into a paste with white spirit in a laboratory mixer (about 70% solids content).

### EXAMPLE 2

Grinding as in Example 1, but with shot having a mean diameter <6 μm and a grinding time of 23 h.

### EXAMPLE 3

5.0 kg of steel balls (diameter: 1.1 mm, weight 5.5 mg), 160 g of white spirit, 150 g of aluminum shot (mean diameter <8 μm) and 6 g of oleic acid are placed in a barrel mill (length: 32 cm, width: 19 cm). Subsequently, the mixture is ground for a period of 16 h at 60 rpm. The product is separated by adequately rinsing white spirit from the grinding balls during screening and is subsequently screened in a wet screening process on a 25 μm screen. The fines are substantially freed from white spirit by means of a suction filter and subsequently made into a paste with white spirit in a laboratory mixer (about 70% solids content).

### COMPARATIVE EXAMPLE 4

### Metalure L 55300 (Eckart)

### COMPARATIVE EXAMPLE 5

### MH 8801 (Asahi)

### COMPARATIVE EXAMPLE 6

### MH 9901 (Asahi)

### COMPARATIVE EXAMPLE 7

### VP 53534 (Eckart), Silver Dollar Pigment

### COMPARATIVE EXAMPLE 8

### MEX 2192 (Eckart), Silver Dollar Pigment

The samples of the examples of the invention and of the selected comparative examples were characterized by water coverages, and mean thicknesses were calculated therefrom.

Determination of the water coverage was carried out on the lines of DIN 55923. Here, in addition to this standard, which only applies to leafing pigments, nonleafing pigments were converted prior to spreading into leafing pigments by the following treatment: 200 mg of aluminum pigments are weighed in the form of a paste or filter cake and dispersed in a solution of 3.15 g of stearic acid and 63 ml of white spirit and treated for 12 min in an ultrasonic bath. Subsequently, the dispersion is filtered off with suction on a glass frit, washed three times with acetone, sucked dry and dried in a vacuum desiccator. In the normal case, about 4 mg of the aluminum powder lubricated with stearic acid are then made into a paste with a glass rod on a watch glass with a few drops of n-butanol and then the entire paste is added to the spreading trough.

In the case of the very thin aluminum pigments of the invention, however, a peculiarity arose in the sample preparation. Here, the water coverages were so high that the metal film was too large for the water trough specified by DIN 55923. It was only possible to achieve appropriately dimensioned metal mirrors by weighing an amount of sample of 1 mg. In this case, however, a marked variance (>10%) was manifested in the measurements, which is caused by the resultant weighing error.

Alternatively, in the case of these samples containing aluminum powder lubricated with stearic acid a dispersion in n-butanol was prepared. The concentration was dimensioned such that 1 ml of this dispersion contained about 1 mg of pigment. For sampling, about 1 ml of dispersion was taken by means of a graduated pipette with vigorous stirring to avoid settling movements of the pigment particles, and the dispersion was carefully dispersed over the spreading trough. The relative variance of the water coverages using this method was less than 10%.

The samples of Examples 1 to 3 of the invention and the selected comparative examples were characterized for a closer determination of the particle thicknesses using a field ion scanning electron microscope.

The samples were prepared for the SEM investigation as described below:

A) Aluminum Pigments of the Invention and Conventional Pigments Obtained by Wet Grinding

The aluminum pigments of the invention or the conventional aluminum pigments obtained by conventional wet grinding are in each case present in the form of a paste or filter cake and are each first washed with acetone and then dried.

A resin customarily used in electron microscopy, for example TEMPFIX (Gerhard Neubauer Chemikalien, D-48031 Munster, Germany), is applied to a sample plate and heated to softening on a hotplate. Subsequently, the sample plate is taken from the hotplate and the respective aluminum powder is scattered onto the softened resin. The resin becomes solid again as a result of cooling and the scattered aluminum pigments—due to the interplay between adhesion and the force of gravity—can be prepared standing almost vertically and fixed to the sample plate. As a result, the pigments can readily be measured laterally in the electron microscope. In the measurement of the thickness, the azimuthal angle α of the pigment is estimated relative to a plane normal to the surface and allowed for when evaluating the thickness according to the formula
h_{eff}=hₘₑₛ/cos α.

The cumulative breakthrough curve was plotted from the h_{eff} values with the aid of the relative frequencies of occurrence. At least about 100 particles are counted.

b) PVD Pigments

A PVD pigment suspension washed a number of times with a large excess of acetone in order to substantially free it from residues of the release coat. Subsequently, the PVD pigments were dispersed in acetone and a drop of the dispersion was distributed on a microscope slide. After the evaporation of the solvent, the slide was sliced. The individual slices can be mounted standing vertically in the electron microscope. With sharp broken edges, sufficient PVD pigments can be measured. Here, on account of the narrow distribution of thicknesses, about 50 particles suffice to obtain meaningful results.

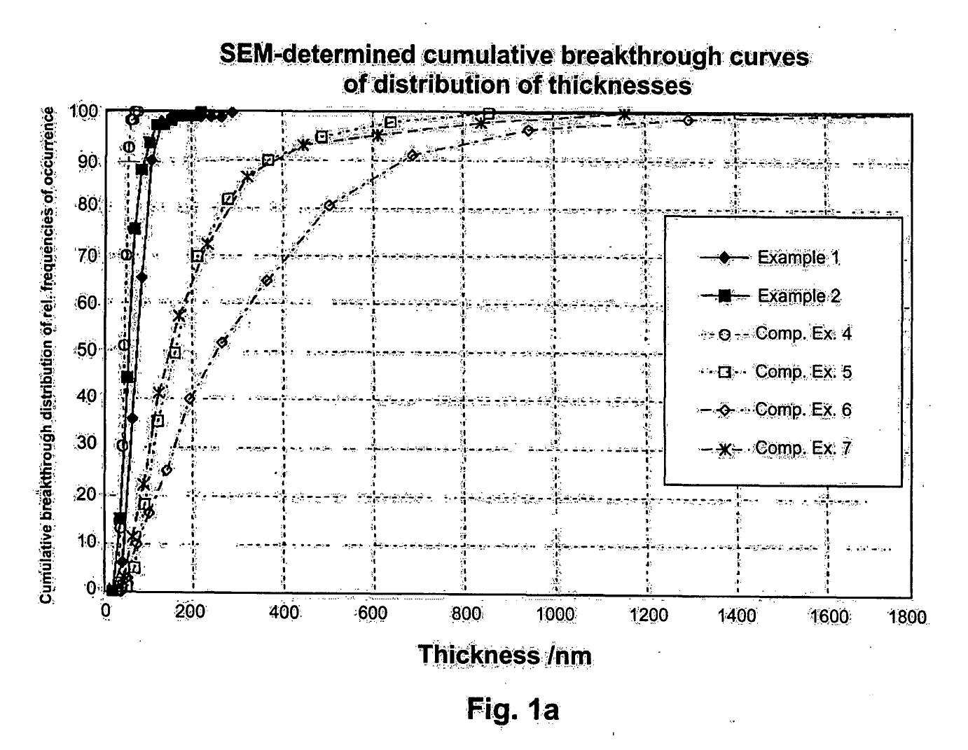

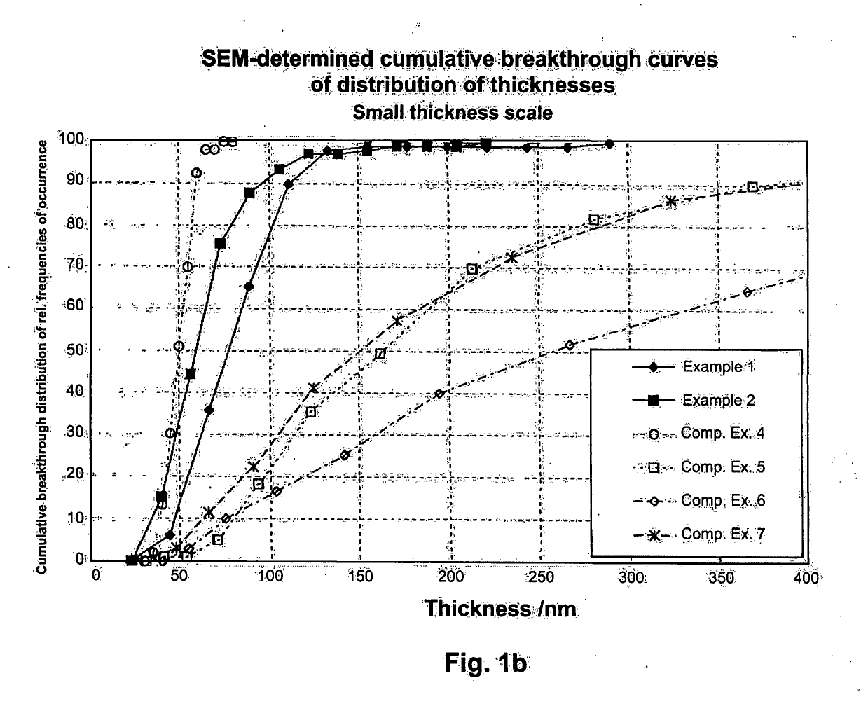

The cumulative breakthrough curves of the distribution of thicknesses of the various samples of the invention and comparison samples are shown in FIGS. 1a and 1b. The curves in FIG. 1b show an enlarged section of the curves in FIG. 1a. The number of particles measured was 50 (PVD pigments) to 192 (conventional pigments). Statistical analyses showed that the cumulative breakthrough curve was substantially constant when from 75 to 100 particles were counted for the pigments of the invention and conventional pigments obtained by wet grinding.

The results are listed in Table 1

TABLE 1
Physical Characteristics

| | | | Mean | SEM | | | | | |
|---|---|---|---|---|---|---|---|---|---|
| | Cilas: | | thickness | statistics | | | | | aspect |
| | d₅₀ | Water coverage | h_{spread} | h₅₀ | h₉₀- | BET | Active Al | Roughness value | ratio |
| Sample | [μm] | [cm²/g] | [nm] | [nm] | h₁₀/h₅₀ | [m²/g] | content | (spread) | (spread) |
| Example 1 | 19 | 50,100 | 80 | 78 | 84% | 6.6 | 92.2% | 0.69 | 238 |
| Example 2 | 19 | 84,600 | 47 | 60 | 97% | 7.6 | 91.1% | 0.45 | 402 |
| Example 3 | 22 | 52,900 | 76 | 79 | 104% | 7.2 | 92.8% | 0.68 | 291 |
| Comparative | 12.5 | — | — | 49 | 41% | — | — | — | 255 |
| Example 4 | | | | | | | | | |
| Comparative | 17 | 30,000 | 133 | 163 | 180% | | — | | 128 |
| Example 5 | | | | | | | | | |
| Comparative | 22 | 24,800 | 161 | 259 | 221% | | — | | 137 |
| Example 6 | | | | | | | | | |
| Comparative | 17.5 | 31,700 | 126 | 148 | 210% | 4.5 | 96.2% | 0.71 | 139 |
| Example 7 | | | | | | | | | |
| Comparative | 14.5 | 20,700 | 193 | — | — | 5.6 | 94.8% | 1.35 | 72 |
| Example 8 | | | | | | | | | |

Example 1: invention
Example 2: invention
Example 3: invention
Comparative Example 4: Metalure L55350 (Eckart) PVD-Pigment
Comparative Example 5: MH 8801 (Asahi)
Comparative Example 6: MH 9901 (Asahi)
Comparative Example 7: VP 53534 (Eckart), silver dollar pigment
Comparative Example 8: MEX 2192 (Eckart), silver dollar pigment

Generally, there is good agreement between the mean thickness h_{spread} determined by the water coverage method and the median value of the cumulative breakthrough curve of the distribution of thicknesses h₅₀. The BET surface areas, d₅₀ values of the size distribution, and the calculated roughness values R and aspect ratios f of the pigments are likewise used. For the calculation of these values—as is customary—the water coverages or average thicknesses calculated therefrom were used.

The longitudinal extent d was determined with the aid of a laser granulometer (Cilas 1064, Cilas, France) and the d₅₀ value of the cumulative breakthrough distribution in μm was chosen as a measure of the mean longitudinal extent in the usual manner.

For the comparative assessment of the pigments of the invention with conventional pigments, the pigments were spread with a doctor blade in a series of concentrations in a conventional nitrocellulose lacquer (Erco Bronzemischlack 2615e; obtainable from Rohm and Haas Germany, Werk Strullendorf, Reinhard-Reichnow-Str. 4, D-96129 Strullendorf) on black/white contrast paper (doctor blade gap: 36 μm) and dried for 24 h at room temperature.

These coatings were characterized optically on the one hand by a gloss measurement at in each case 20° and 60° following DIN 67 530 (apparatus: micro-TRI gloss produced by Byk-Gardner, D-82538 Geretsried, Germany). The apparatus was calibrated here by means of dark calibration and a black mirror glass plate with values of 92 for 20° and 95 for 60°. On the other hand, a colorimetric determination of the brightness values L* was characterized at a constant irradiation angle of 45° with different observation angles (relative to the gloss angle) (apparatus: Multiflash M 45, Optronics).

From the brightness values at 15°, 45° and 110°, according to a formula originally given by DuPont, a flop index can be determined which reproduces well the angle-dependent brightness change of conventional metallic coatings (A. B. J. Rodriguez, JOCCA, (1992(4)) pp. 150-153):

$$flop index = 2 \text{,} 69 \times \frac{{( {L}_{15 °}^{*} - {L}_{110 °}^{*} )}^{1 , 11}}{{( {L}_{45 °}^{*} )}^{0 , 86}}$$

For the assessment of the covering power of the coatings, the ratio of the brightnesses L* at an observation angle of 110° on black to white undercoat was used. If this ratio was >0.98, the coating is designated as having a full covering capacity. Measurement at such a low observation angle is particularly sensitive, since when the undercoat is not fully covered, the measurement is recorded virtually “between” the mainly plane-parallel oriented metal pigments. The agreement with the visual impression of the observer is very good. In the literature, on the other hand, the brightness differences are usually assessed by diffuse measurement (for example in EP 0 451 785), which, however, leads to a considerably lower sensitivity and, moreover, does not agree with the visual impression.

The covering power of the coatings depends decisively on the concentration of the aluminum pigments. As a measure of this concentration, a surface area concentration of aluminum pigments c_{Al} was calculated in mg/cm². This variable is calculated by means of the wet lacquer layer thickness, which is predefined by the doctor blade, according to the following formula:

$${c}_{Al} = 0 \text{,} 1 * \frac{{m}_{Al}}{{m}_{wet lacquer , Al}} * {ρ}_{wet lacquer , Al} * RH [ mg / {cm}^{2} ]$$

in which:
m_{Al}: is the weight of the aluminum pigments
m_{wet lacquer,Al}: is the weight of the wet lacquer and of the aluminum pigments including the solvent for the aluminum pigments in the paste (as a rule 10.0 g)
ρ_{wet lacquer,Al}: is the density of the wet lacquer mixed with aluminum pigment
RH: is the doctor blade gap in μm.

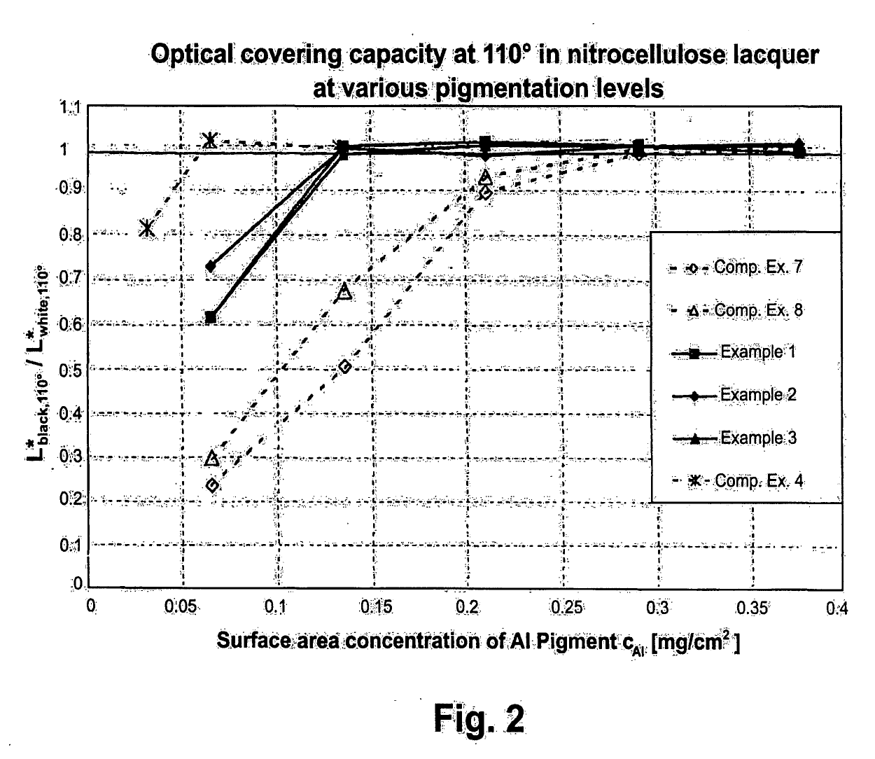

In FIG. 2, the optical covering power criterion is plotted against the calculated surface area concentration of aluminum pigment for selected examples.

It can be clearly seen from FIG. 2 that the aluminum pigments of the invention have a significantly higher covering power than the conventional aluminum pigments. With the aid of these data, that surface area concentration was determined by interpolation at which the ratio L*_{110°,black}/L*_{110°,white} was 0.98.

Examples 1 to 3 of the invention exhibit excellent covering power at a surface area concentration of less than 0.15 mg/cm². Only the PVD pigments prepared by means of the more expensive PVD process (Comparative Example 4) show a better covering power.

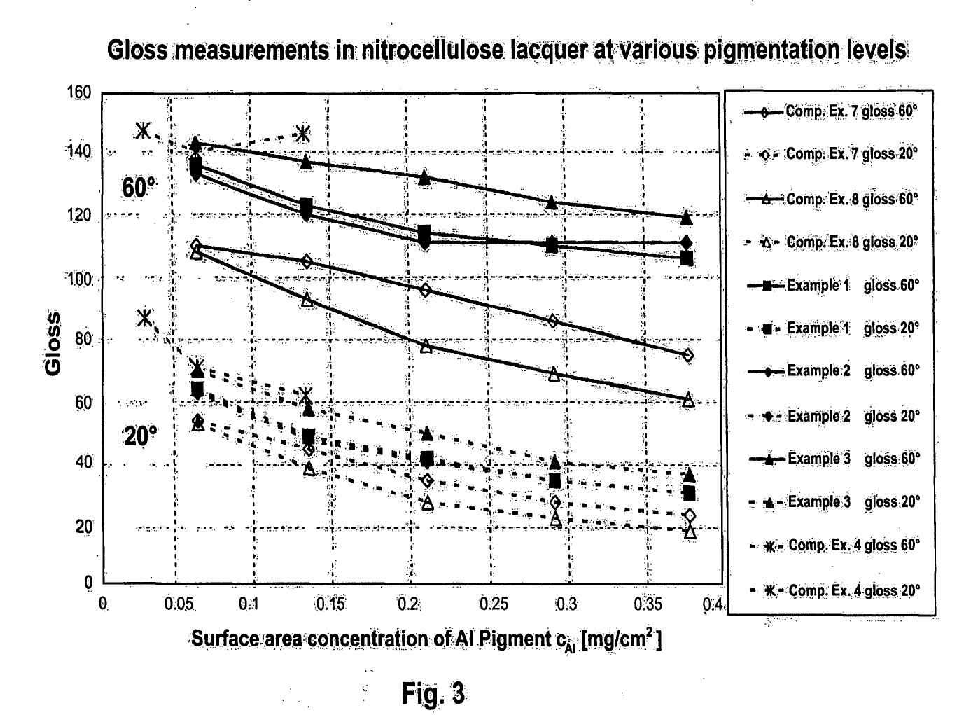

In FIG. 3, the gloss values measured in each case at a 60° and 20° incidence/emergence angle geometry of the coatings are plotted against the surface area concentration of aluminum pigment. The gloss decreases nearly linearly with increasing surface area concentration. This is to be attributed to the increasingly poor orientation of the metal pigments with increasing pigmentation level. Poorer orientation is caused here by an increase in the errors in stacking of the pigments within the lacquer layer. This effect is particularly pronounced here, since the coating shows a large Al/binder ratio on account of the low solids content of the lacquer (nonvolatile fraction: about 6%).

It can consequently be seen from FIG. 3 that—apart from the PVD pigment Metalure® (Comparative Example 4)—the aluminum pigments of the invention (Examples 1 to 3) exhibit markedly better gloss values at all surface area concentrations than conventional aluminum pigments (Comparative Examples 7 and 8).

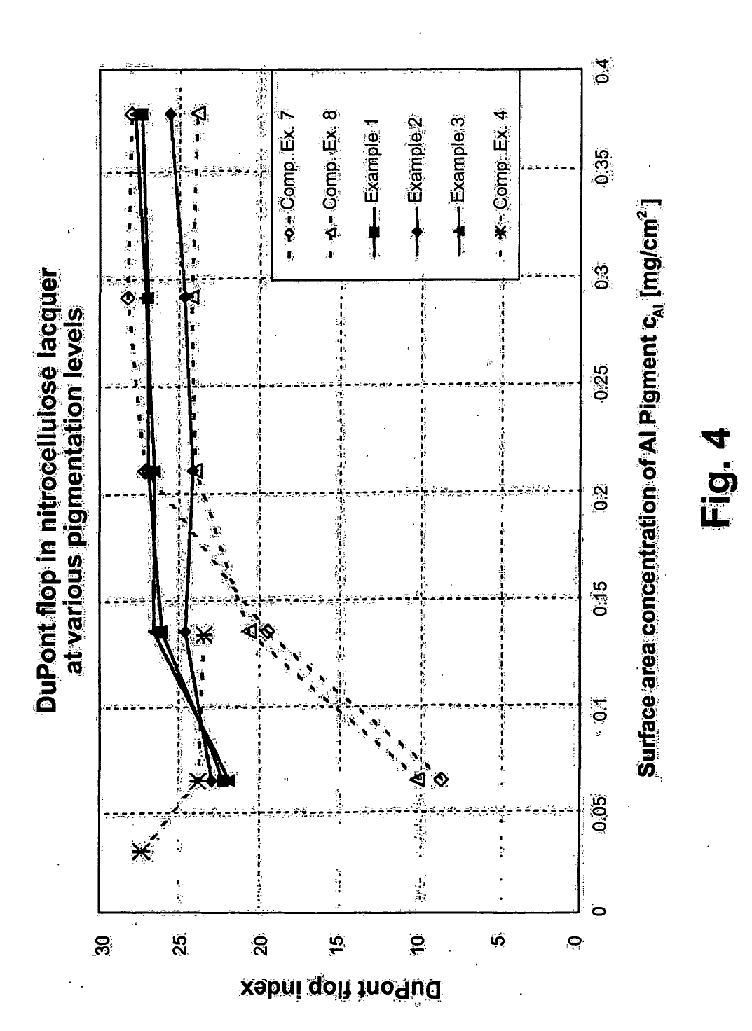

In FIG. 4, the flop values according to DuPont are plotted against the surface area concentration of aluminum pigment. At high pigmentations, the flop values of the pigments of the invention and of the conventional pigments are comparable. But distinct differences are apparent below about 0.15 mg/cm², at which values the covering power of the conventional pigments is already very poor. On account of their far greater covering power, the aluminum pigments of the invention can be particularly advantageously employed at low pigmentation levels at which conventional pigments no longer have any covering capacity. Here, one has access to coatings showing very high gloss, good flop, and an intensely metallic appearance, i.e. a very good chrome effect.

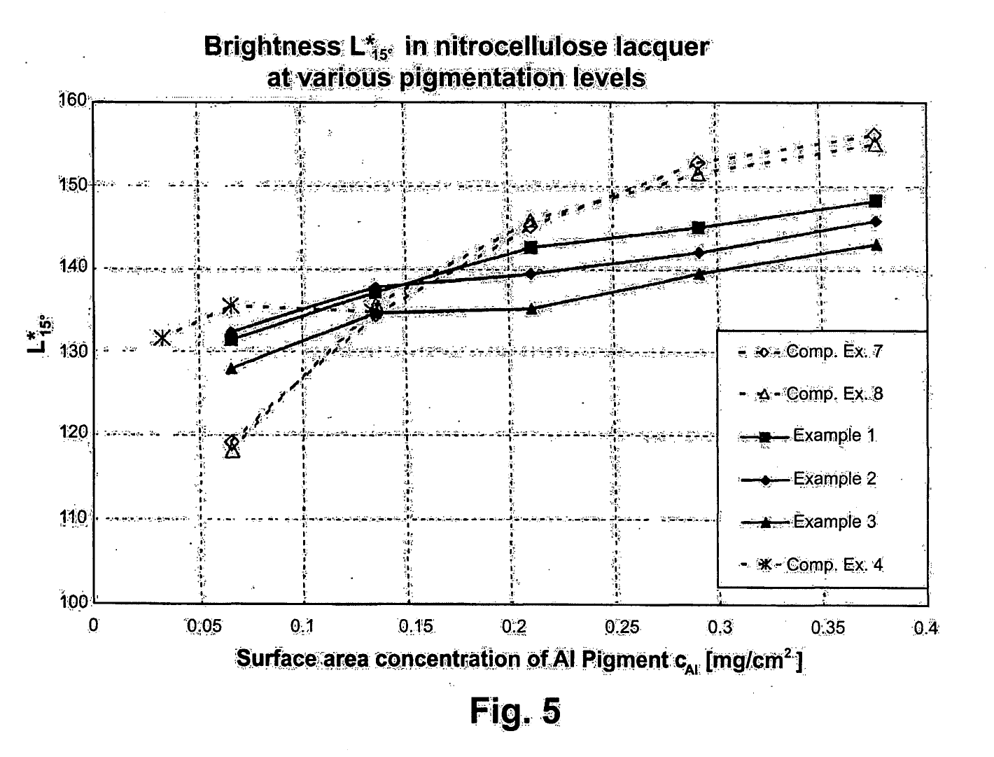

In FIG. 5, as a customary calorimetric measure of the brightness, the L* values at 15° are plotted against the surface area concentration of aluminum pigment. At high pigmentation levels the values of the conventional pigments first predominate but drop considerably with increasing loss of covering capacity.

This result of calorimetric measurements contradicts the visual impression, however. The observer assigns the coatings of the pigments of the invention a markedly higher brightness than those of the conventional pigments.

It is suspected that this discrepancy can be explained as follows:

On account of their low thickness, the pigments of the invention show, similarly to PVD pigments, extremely good orientation and thereby a very high direct reflection, i.e. a high degree of gloss, such that even at an observation angle of 15°, the fraction of the scattered light near the gloss angle is already low. Therefore a lower brightness is suggested in the colorimetric assessment. This, however, contradicts the visual impression. Coatings of conventional pigments (Comparative Examples 7 and 8) on the contrary show a markedly “whiter” or “milkier” brightness. The pigments of the invention, on the other hand,—as also PVD pigments (Comparative Example 4)—show a considerably greater characteristic metallic effect. Accordingly, the flop of the pigments of the invention is also to be rated somewhat higher than values determined by the DuPont flop index suggest. This also corresponds to the visual impression of the coatings.

Such misleading by the colorimetric data may possibly also underlie a relationship claimed according to the teaching of EP 0 451 785 between a loss of brightness with increasing water coverages and thus with increasing covering power. In FIG. 1 of said patent specification, there is claimed to be a maximum of the brightness L at a water coverage of 3.2 m²/g, which suggests a marked drop at water coverages higher than 5 m²/g in particular. The brightness is not related, however, to a measuring angle, but on the contrary appears to have been measured diffusely. It was overlooked in this case that thinner pigments on account of the higher direct reflection of incident light must necessarily possess a lower content of scattered light.

Tab. 2 lists the critical covering power surface area concentrations determined from FIG. 2 and the interpolated colorimetric data at this concentration are shown.

TABLE 2
Optical data for covering power surface area concentrations of Al
Pigment C_{Al,98%}

| | Covering power | | | | | |
|---|---|---|---|---|---|---|
| | surface area | | | DuPont | Brightness | |
| | concentration of Al | Gloss | Gloss | flop index | L*15° | General |
| | Pigment C_{Al,98%} | 20° with | 60° with | with | with | visual |
| Sample | [mg/cm²] | C_{Al,98%} | C_{Al,98%} | C_{Al,98%} | C_{Al,98%} | impression |
| Ex. 1 of | 0.134 | 49 | 124 | 25.8 | 137 | very metallic. |
| invention. | | | | | | chrome effect |
| Ex. 2 of | about 0.12 | 52 | 123 | 24.2 | 136 | very metallic. |
| invention | | | | | | chrome effect |
| Ex. 3 of | 0.135 | 59 | 137 | 26.4 | 134 | very metallic. |
| invention | | | | | | chrome effect |
| Comp. | 0.29 | 36 | 87 | 28.2 | 153 | metallic |
| Ex. 7 | | | | | | |
| Comp. | 0.27 | 25 | 72 | 27.1 | 150 | metallic. |
| Ex. 8 | | | | | | fairly “white” |
| Comp. | about 0.05 | 79 | 144 | 25.4 | 134 | Extremely |
| Ex. 4 | | | | | | metallic |
| | | | | | | chrome effect |

A higher gloss of the pigments of the invention was also found in wet lacquer coatings. Tab. 3 lists the calorimetric data of wet lacquer coatings of selected examples. Without exception, the pigments prepared according to Example 1 of the invention have a higher gloss than conventional pigments obtained by conventional wet grinding. However, the gloss is not as high as with PVD pigments (see Comparative Example 4).

The coatings in Tab. 3 were produced by spraying “to coverage”, i.e. covering pigmentation levels were determined in a series of concentrations. The pigmentation levels used in each case (based on the lacquer formulation) and the measured layer thicknesses of the coatings are indicated. The layer thicknesses were measured with a Qua Nix 1500 (Lau GmbH, D-58675 Hemer, Germany). Also clear here is the higher covering power and thus the associated lower layer thicknesses of the pigments of the invention in comparison with conventional pigments. Here too, however, a PVD pigment has an even better covering power and more intense metallic properties.

TABLE 3
Wet lacquer coatings without clear lacquer at various layer
thicknesses and pigmentation levels sprayed to coverage.
Lacquer system: 2K chrome effect lacquer having a low solids content
(Eckart “Metalure” pamphlet).

| | | | | Pigmentation | |
|---|---|---|---|---|---|
| | Gloss | | DuPont flop | (based on | Layer |
| Sample | 60° | 20° | index | formulation) | thickness |
| Example 1 of inv. | 84 | 29 | 24.0 | 1.8% | 4-6 μm |
| Metalure L 55700 | 128 | 57 | 24.6 | 1.5% | 2-3 μm |
| (Comp. Ex. 4) | | | | | |
| VP 53534 | 74.4 | 25 | 24.5 | 4% | 6-8 μm |
| (Comp. Ex. 7) | | | | | |

Examples of the Passivation of the Aluminum Pigments of the Invention:

### EXAMPLE 9

### SiO₂-Coated Aluminum

55.1 g of a paste containing aluminum pigments according to Example 1 (equivalent to 38.5 g of Al) are dispersed in 375 ml of isopropanol and brought to boiling temperature. 13.35 g of tetraethoxysilane are added. Subsequently, a solution of 5.4 g of 25% strength NH₃ in 9.3 g of water is metered in over a period of 3 h. After a further 3 h, the mixture is cooled to room temperature and the suspension is filtered off with suction by means of a Buchner funnel. Subsequently, the product is dried overnight in a vacuum drying oven at 100° C.

### EXAMPLE 10

### Chromated Aluminum

18 g of a chromic acid solution are prepared by dissolving 4.5 g of CrO₃ in 13.5 g of water (demineralized).

220 g of water (demineralized) are heated to 90° C. in a reactor having a capacity of 1 liter. With vigorous stirring (stirring unit: Stollenscheibe), first 21 g of butyl glycol are added and subsequently 125 g of the aluminum pigments described in Example 1 in the form of a white spirit paste with a solids content of 70%. A few minutes later, the chromic acid solution is added at a reaction temperature of 80° C. The mixture is allowed to react for a further 50 min with vigorous stirring. The reaction mixture is then allowed to cool for 30 min and decanted in a beaker a number of times with 250 ml of a 5% strength of a demineralized H₂O/butyl glycol solution each time until yellow coloration of the supernatant solution no longer occurs. Subsequently, the product is filtered off in a suction filter and washed with a copious amount of water (about 3 liters).

Gassing Test:

8.6 g of Al are incorporated in the form of a paste into 315 g of colorless water-mix lacquer (ZW42-1100, BASF Würzburg) and brought to pH 8.2 with dimethanol-ethanolamine. 300 g of this lacquer are filled into a gas wash bottle and this is closed with a double-chamber gas bubble counter. The amount of gas can be read off by means of the amount of water displaced in the lower chamber of the gas bubble counter. The gas wash bottle is temperature-controlled at 40° C. in a water bath and the test is carried out over 30 days. It is regarded as passed if after 7 days not more than 4 ml and, after 30 days, not more than 20 ml of hydrogen were evolved.

TABLE 4
Results of gassing test on coated thin aluminum pigments

| Sample | Gassing for 7 d | | Gassing for 30 d |
|---|---|---|---|
| Example 9 | 2 | ml | 8 ml |
| Example 10 | 1 | ml | 5 ml |
| Comparative Example 11 | <3 | h !! | — |
| (uncoated pigment of | | | |
| Example 1) | | | |

d = days

It can be seen from Table 4 that the aluminum pigments of the invention can be excellently stabilized against corrosion.

The process of the invention makes it possible to produce aluminum pigments which in their physical properties come very close to the PVD pigments, but can be prepared in a significantly simpler manner. Compared with the conventional aluminum pigments, the aluminum pigments of the invention exhibit, in particular with respect to their covering power and their gloss, markedly improved properties. Finally, the aluminum pigments of the invention do not show any tendency to agglomerate, which is a drawback of PVD pigments. The aluminum pigments of the invention can therefore be used in much more concentrated form in preparations to be added, for example, to lacquer systems, printing inks, or cosmetics. This facilitates handling significantly.

The aluminum pigments of the invention therefore combine the advantageous properties of conventional aluminum pigments, in particular their ease of manufacture and handling, with those of PVD pigments, in particular their high covering power, high gloss properties and intensely metallic appearance.

What is claimed is:

1. An aluminum pigment, which is at least partially coated with a lubricant, characterized in that said aluminum pigment has
a) a water coverage between 40,000 and 130,000 cm²/g,
b) a mean thickness h of less than 100 to 30 nm as calculated from the water coverage and the h₅₀ value as determined from the cumulative breakthrough curve of a scanning electron microscope thickness count,
c) as determined by a scanning electron microscope thickness count, a relative width of the distribution of thicknesses Δh of from 70% to 140%, as calculated on the basis of the corresponding cumulative breakthrough curve of the relative frequencies of occurrence, according to the formula
$$Δ h = 100 \times \frac{{h}_{90} - {h}_{10}}{{h}_{50}} .$$
d) an aspect ratio d₅₀/h of more than 200, and
e) a roughness value of from 0.30 to 0.9, as calculated from the specific surface area as determined by the BET test method and the water coverage, according to the formula:
BET value/2×water coverage.

2. The aluminum pigment as defined in claim 1, characterized in that said aluminum pigment has, as determined by a scanning electron microscope thickness count, a relative width of the distribution of thicknesses Δh of from 75% to 120%, as calculated on the basis of the corresponding cumulative breakthrough curve of the relative frequencies of occurrence according to the formula
$$Δ h = 100 \times \frac{{h}_{90} - {h}_{10}}{{h}_{50}} .$$

3. The aluminum pigment as defined in claim 1, characterized in that said aluminum pigment has an aspect ratio d₅₀/h of more than 220.

4. The aluminum pigment as defined in claim 1, characterized in that said aluminum pigment has a roughness value, calculated from the specific surface area, as determined by the BET test method, and the water coverage, according to the following formula: BET value/2×water coverage of 0.35 to 0.9.

5. The aluminum pigment as defined in claim 1, characterized in that said aluminum pigment is at least partially coated with a fatty acid as lubricant.

6. The aluminum pigment as defined in claim 1, characterized in that said aluminum pigment is at least partially coated with stearic acid as lubricant.

7. The aluminum pigment as defined in claim 1, characterized in that said aluminum pigment is at least partially coated with oleic acid as lubricant.

8. The aluminum pigment as defined in claim 1, characterized in that said aluminum pigment is at least partially coated with a mixture of stearic acid and oleic acid as lubricant.

9. The aluminum pigment as defined in claim 1, characterized in that said aluminum pigment is at least partially coated with a phosphonic acid, a phosphoric acid ester or a mixture thereof as lubricant.

10. The aluminum pigment as defined in claim 1, characterized in that said aluminum pigment is coated with a passivating inhibitor or anti-corrosion layer.

11. The aluminum pigment as defined in claim 10, characterized in that said passivating inhibitor layer comprises corrosion inhibiting organic phosphonic acids or phosphoric acid esters, functional organic silanes, aliphatic or cyclic amines, aliphatic or aromatic nitro compounds, oxygen-, sulfur- or nitrogen-containing heterocyclics, sulfur- or nitrogen-containing higher ketones, aldehydes and alcohols, thiols, β-ketoesters, β-diketones, or mixtures thereof.

12. The aluminum pigment as defined in claim 10, characterized in that said passivating anti-corrosion layer comprises silicon oxide, zirconium oxide, aluminum oxide, chromium oxide, polymerized plastic resins, vanadium oxides, molybdenum oxides or peroxides, phosphates, phosphites, borates or mixtures thereof.

13. The aluminum pigment as defined in claim 10, characterized in that said passivating anti-corrosion layer comprises silicon dioxide.

14. The aluminum pigment as defined in claim 1, characterized in that said aluminum pigment has been oxidized by water in an aqueous chemical process and said aluminum pigment has modified color.

15. The aluminum pigment as defined in claim 1, characterized in that said aluminum pigment is a powder or a compacted form.

16. A process for the production of a pigment as defined in claim 1, comprising the following step: a) milling of aluminum particles to an aluminum pigment within a milling device in the presence of solvent, lubricants and milling media having an individual weight of from 2 to 13 mg, over a time period between 15 and 72 hours.

17. The process as defined in claim 16, characterized in that said milling media have an individual weight of from 5.0 to 12 mg.

18. The process as defined in claim 16, characterized in that said aluminum pigment is subjected to a size classification in an additional step b).

19. The process as defined in claim 16, characterized in that said aluminum pigment prepared in step a) or step b) is converted to a compacted form.

20. The process as defined in claim 16, characterized in that said aluminum pigment prepared in step a) or step b) is converted to powdered aluminum.

21. The process as defined in claim 16, characterized in that the solvent used is an organic solvent.

22. The process as defined in claim 16, characterized in that the solvent used is water and the lubricant used is selected from the group consisting of an organic phosphonic acid, an ester thereof, a phosphoric acid, an ester thereof, and mixtures thereof.

23. (canceled)

24. (canceled)

25. A nail varnish composition, characterized in that said nail varnish contains an aluminum pigment as defined in claim 1.

26. A water based paint composition, characterized in that said water based paint contains an aluminum pigment as defined in claim 9.

27. A coating composition comprising the aluminum pigment as defined in claim 1.

28. A paint composition comprising the aluminum pigment as defined in claim 1.

29. A printing ink composition comprising the aluminum pigment as defined in claim 1.

30. A powder coating composition comprising the aluminum pigment as defined in claim 1.

31. A plastic composition comprising the aluminum pigment as defined in claim 1.

32. A security printing ink composition comprising the aluminum pigment as defined in claim 1.

33. A ceramic composition comprising the aluminum pigment as defined in claim 1.

34. A cosmetic formulation composition comprising the aluminum pigment as defined in claim 1.

35. A water-based paint composition comprising the aluminum pigment of claim 10.

36. A coating composition for exterior applications comprising the aluminum pigment of claim 10.

37. The aluminum pigment as defined in claim 13, wherein the silicon dioxide surface is coated with silanes.

38. The aluminum pigment as defined in claim 15, wherein said aluminum pigment is a non-dusting powder.

39. The aluminum pigment as defined in claim 15, wherein said compacted form is a paste, granules, or pellets.

40. The process as defined in claim 19, wherein the compacted form is a paste, granules, or pellets.

41. The process as defined in claim 20, wherein the powdered aluminum is a non-dusting aluminum powder.

42. The process as defined in claim 21, wherein the organic solvent comprises white spirit, solvent naphtha, isopropanol, an alcohol, a ketone, or a mixture thereof.
